(19) United States
(12) Patent Application Publication
(10) Pub. No.: US 2015/0290117 A1
(43) Pub. Date: Oct. 15, 2015

(54) SENSORILY ATTRACTIVE HYDRODISPERSION COMPRISING WAXES

(71) Applicant: BEIERSDORF AG, Hamburg (DE)

(72) Inventors: Cecile Ratschow, Hamburg (DE); Christiane Meyer, Hamburg (DE); Janina Uhlen, Hamburg (DE); Heike Lerg, Hamburg (DE); Bente Nissen, Hamburg (DE)

(21) Appl. No.: 14/443,862

(22) PCT Filed: Nov. 12, 2013
(86) PCT No.: PCT/EP2013/073586

§ 371 Date: May 19, 2015

(30) Foreign Application Priority Data
Nov. 20, 2012 (DE) 10 2012 221 227.2

(51) Int. Cl. A61K 8/92; A61Q 19/00; A61K 8/81

(52) U.S. Cl. CPC A61K 8/92; A61K 8/8147; A61Q 19/00

(57) ABSTRACT

The invention relates to a hydrodispersion, comprising thickeners, waxes, and oils. The waxes and the overall preparation have specific melting ranges that ensure a pleasant, sensorily light profile and good care effects.

The invention encompasses hydrodispersion comprising thickeners, waxes and oils. The waxes and the overall formulation have specific melting ranges which ensure a pleasant, sensorily light profile and good care effects.

Common manifestations of cosmetic or dermatological formulations are finely dispersed multiphase systems in which one or more fat or oil phases are present alongside one or more water phases. Among these systems, in turn, the actual emulsions are the most common.

Also known are low-emulsifier or emulsifier-free formulations based on what are called hydrodispersions. Hydrodispersions are dispersions of a liquid, semisolid or solid inner (discontinuous) lipid phase in an outer aqueous (continuous) phase. Hydrodispersions—like emulsions too, which feature a similar phase arrangement—are metastable systems and therefore have a tendency to be converted to a state with two intrinsically coherent discrete phases. In a conventional 0/W emulsion, the choice of a suitable emulsifier prevents phase separation. In contrast to conventional emulsions, hydrodispersions, however, contain only very small amounts of emulsifier (up to 2% by weight) or may even be entirely free of emulsifiers.

In the case of hydrodispersions which consist of a liquid lipid phase in an outer aqueous phase, the stability of the multiphase system is typically assured by forming a gel structure with the lipid droplets in stable suspension therein in the aqueous phase with the aid of a gel former or thickener.

Known thickeners are homopolymers or copolymers of acrylic acid and/or acrylamide and derivatives thereof. However, polyacrylate-based formulations frequently do not exhibit any lasting care effects. Gel formulations are also salt-sensitive, and so they frequently break as a result of the presence of salts on application to the skin.

Furthermore, hydrodispersions often feel tacky compared to emulsions. A further disadvantage of hydrodispersions, especially of those containing acrylates as gel formers, is the disadvantageous abrasion when the hydrogel layer applied to the skin is removed easily and in an unappealing manner (“roll formation”).

A further disadvantage of the prior art hydrodispersions is that they have a tendency to phase separation in the case of a relatively high content of lipids (greater than 4% by weight), and this can occur especially at relatively high temperatures (≧40° C.).

WO 2005097057 A1 discloses gel formulations comprising polymeric gel formers, water, oils and at least one wax having a melting point of 30° C. or more. The formulations are free of cationic or anionic emulsifiers or surfactants.

WO 2005097055 A1 describes gel formulations comprising polymeric gel formers, water, an oil phase which is liquid at 25° C. and at least one wax component having a melting point of at least 30° C., selected from the group of the pentaerythritol esters, the dipentaerythritol esters and/or the tripentaerythritol esters.

However, the organoleptic properties of these gel formulations are not found to be especially attractive. The residue has a very waxy feel.

It is therefore desirable to provide hydrodispersions having improved organoleptic properties and especially having a residue with a less waxy appearance.

Cosmetics can be summarized as all measures which, for esthetic reasons, make changes to the skin and hair or are used for personal care. Cosmetics thus means taking care of, improving and beautifying the outward appearance of the body, in order to please other people and oneself in a visible, tangible and olfactorily perceptible manner. For millennia, cosmetics have been used by humans for this purpose. The lips and face were colored, valuable oils were used as ointments and fragrant water was used for bathing.

A property of cosmetic products which is very important to the consumer but can be measured quantitatively only with difficulty is their texture. The term “texture” is understood to mean those properties of a cosmetic which derive from the structure of the formulation, are perceived by the sense of touch and in some cases can be expressed in terms of mechanical or rheological flow properties. Texture can be tested especially by means of organoleptic methods. The texture of cosmetic products, which can be influenced in some cases with the aid of additives, is nearly as important to the consumer as the effects thereof that can be detected objectively.

The term “organoleptics” refers to the scientific discipline concerned with the assessment of cosmetic formulations on the basis of sensory impressions. The organoleptic assessment of a cosmetic is made on the basis of the visual, olfactory and tactile impressions.
Visual impressions: all features perceptible to the eye (color, shape, structure).
Olfactory impressions: all odor impressions perceptible on intake of air through the nose, which can frequently be differentiated into initial odor (top note), main odor (middle note, body) and final odor (finish). Even the volatile substances not released until application contribute to the olfactory impression.
Tactile impressions: all impressions from the sense of touch, which relate principally to the structure and consistency of the product.

Organoleptic analysis makes use of the possibility of gaining an integrated organoleptic overall impression of a product. Disadvantages of organoleptic analysis are the subjectivity of the impression, the ease with which test subjects can be influenced and the resultant significant scatter in the results. These weaknesses are nowadays countered by the use of groups of trained test subjects, screening of the testers from one another, and statistical evaluation of the usually numerous analytical data.

The problem addressed was that of providing cosmetic or dermatological hydrodispersions which are formulated with waxes, have a pleasant, sensorily light profile and good care effects, and do not break on application to the skin.

It has now been found that, surprisingly, the organoleptics of the formulation are positively influenced when the wax combination is selected such that it contains at least one wax component having a melting point Tₒₙₛₑₜ of less than 30° C. and the overall formulation advantageously has at least one melting range by means of DSC between 5° C. and 30° C.

The invention is a hydrodispersion comprising

a.) water, advantageously 60%-95% by weight, especially 70%-85% by weight,
b.) 0.05%-5% by weight of at least one thickener,
c.) 2% to 20% by weight of one or more waxes and
c.) 0% to 20% by weight of one or more oils, especially more than 0.5% by weight, advantageously 1% to 10% by weight, based in each case on the total mass of the dispersion,
wherein at least one wax has a melting point Tₒₙₛₑₜ of <30° C.

The dispersion preferably does not contain any anionic or any cationic emulsifiers and surfactants.

The dispersion advantageously encompasses at least one melting range between 5° C. and 30° C. by DSC. One further melting range or a plurality thereof may be outside this temperature range.

In contrast to the prior art gel formulations, the hydrodispersions of the invention have attractive organoleptic properties and care properties.

The dispersion of the invention has a surprisingly rich texture when dispensed.

Because of the wax melting cascade of the invention, the formulations additionally exhibit a cooling effect on distribution and are very easy to distribute on the skin.

The thickener(s) is/are chosen advantageously in accordance with the invention from one or more of the following groups:
homopolymers or copolymers of acrylic acid and/or acrylamide and derivatives thereof,
organic natural compounds such as, in particular, agar-agar, carrageenan, tragacanth, gum arabic, alginates, pectins, polyoses, guar flour, carob seed flour, starch, dextrins, gelatins, casein, xanthan gum,
organic modified natural substances, for example carboxymethyl cellulose and other cellulose ethers, hydroxyethyl and hydroxypropyl cellulose and microcrystalline cellulose or the like,
organic fully synthetic compounds, for example polyacrylic and polymethacrylic compounds, vinyl polymers, polycarboxylic acids, polyethers, polyimines, polyamides, polyurethanes, AMPS copolymers, for example Ammonium Acryloyl-dimethyltaurate/VP Copolymer and Sodium Acrylate/Acryloyldimethyltaurate/Dimethyl-acrylamide Crosspolymer, Acrylic Acid/VP Crosspolymer, PVP, Acrylamide/Ammonium Acrylate Copolymer.

Particularly preferred thickeners in the context of present invention are polyacrylates, i.e. polymers based on esters of acrylic acid (polyacrylic esters) of the general structural formula

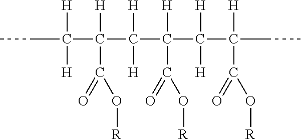

in which R represents linear, branched or cyclic alkyl radicals optionally containing functional substituents (e.g. hydroxyl, amine or epoxy groups), for example methyl, ethyl, isopropyl, tert-butyl, cyclohexyl, 2-ethylhexyl, dodecyl, 2-hydroxyethyl and 2-dimethylaminoethyl.

Very particular preference is given to polyacrylates having the INCI name Sodium Polyacrylate, for example that obtainable under the Cosmedia SP trade name from Cognis.

The proportion of thickeners is preferably chosen within the range from 0.1% to 2% by weight, based on the total mass of the formulation.

If individual waxes or a plurality thereof are used, the stated proportions are based either on individual waxes or a plurality thereof, provided that the total mass does not exceed the advantageous sum total.

The term “wax” is typically understood to mean all natural or synthetic substances and substance mixtures having the following properties:

The waxes of the invention, at room temperature (20° C.), are of solid to brittle, hard consistency, coarse to finely crystalline, and translucent to opaque, and melt above 20° C. without decomposition. They are of low viscosity and do not form threads even slightly above the melting point, and show a strong temperature dependence of consistency and solubility. It is possible in accordance with the invention to use one wax component or a mixture of wax components which start(s) to melt at a temperature Tₒₙₛₑₜ<30° C. but is/are still solid at RT (20° C.). In other words, at least one wax has a melting point Tₒₙₛₑₜ<30° C., or advantageously all the waxes chosen have this melting point.

Tₒₙₛₑₜ is determined by means of DSC.

DSC (differential scanning calorimetry) is a thermal method for measurement of the amount of heat released/absorbed by a sample in isothermal mode, heating or cooling (see DIN 53765, DIN 51007, ASTM E 474, ASTM D 3418). DSC is a comparative test method which enables the determination of amounts of heat in physical and chemical processes. When a material changes its physical state, for example melting or conversion of one crystal form to another or when it reacts chemically, heat is absorbed or released. These amounts of heat can be measured quantitatively with the aid of DSC. The method runs cyclically, such that defined cooling takes place after the first heating curve and then the sample is heated once again within the temperature range specified. Two kinds of information are thus obtained: in the first heating curve, all the thermal effects including the history are apparent. In the second heating curve, the history has been eliminated and the pure thermal characteristics of the sample can be evaluated under defined cooling conditions. The melting temperature Tₒₙₛₑₜ of the waxes is ascertained in the second heating curve. In contrast, the melting range of the hydrodispersion between 5° C. and 30° C. by DSC is the range ascertained in the first heating curve.

Waxes used in accordance with the invention may also be fats and fat-like substances having waxy consistency, provided that they have a Tₒₙₛₑₜ of <30° C. These include fats (triglycerides), mono- and diglycerides, natural and synthetic waxes, fatty alcohols and wax alcohols, esters of fatty alcohols and fatty acids, and fatty acid amides, or any desired mixtures of this substance.

More preferably, the waxes are chosen from the group of fats, especially from:
Shorea stenoptera seed butter (Tₒₙₛₑₜ=14.9° C.),
hydrogenated vegetable oil (Tₒₙₛₑₜ=18.9° C.),
hydrogenated coco-glycerides (Tₒₙₛₑₜ=26.8° C.),
Butyrospermum parkii butter (Tₒₙₛₑₜ=28.3° C.),
Theobroma cacao (cocoa) seed butter (Tₒₙₛₑₜ=14.5° C.),
mango butter (Tₒₙₛₑₜ=19.4° C.),
hydrogenated palm kernel glycerides and hydrogenated palm glycerides (Lipocire A, Gattefossé) (Tₒₙₛₑₜ=20.3° C.),
Acacia decurrens/jojoba/sunflower seed wax polyglyceryl-3 esters (Tₒₙₛₑₜ=14.4° C.),
   from the group of the esters of fatty acids, especially from
cetearyl nonanoate (Tₒₙₛₑₜ=24° C.),
methyl palmitate (Tₒₙₛₑₜ=27.7° C.),
   and from the group of the fatty alcohols, especially from
cetearyl alcohol (Tₒₙₛₑₜ=28.1° C.).

A further aspect essential to the invention is that the overall formulation has at least one melting range between 5° C. and 30° C. by DSC.

Shown below are comparative DSC measurements which illustrate the inventive influence of the melting range of the hydrodispersions.

For this purpose, the following comparative formulations were tested:

A (example 2 from EP1742607 B1)

| INCI | % by weight |
|---|---|
| Coco-Caprylate/Caprate | 5 |
| Dicaprylyl Carbonate | 5 |
| Pentaerythrityl Distearate | 1 |
| Sodium Polyacrylate | 1 |
| Aqua | 88 |

B (example 6 from WO 2005097057 A1)

| INCI | % by weight |
|---|---|
| Cyclomethicone | 10 |
| Pentaerythrityl Distearate | 1 |
| Sodium Polyacrylate | 1 |
| Aqua | 88 |

C (example 4 from EP1742607 B1)

| INCI | % by weight |
|---|---|
| Coco-Caprylate/Caprate | 5 |
| Dicaprylyl Carbonate | 5 |
| Pentaerythrityl Distearate | 1 |
| Sodium Polyacrylate | 1 |
| Aqua | 73 |
| Alcohol Denat. | 15 |

D (Example 1)

| INCI | % by weight |
|---|---|
| Olus Oil | 3.2 |
| Candelilla Cera | 0.2 |
| Cetyl Palmitate | 1 |
| Butyrospermum Parkii Butter | 4 |
| Hydrogenated Vegetable Oil | 0.6 |
| Hydrogenated Coco-Glycerides | 5 |
| Zea Mays Oil + CI 40800 + Tocopherol | 0.0004 |
| Cyclomethicone + Dimethiconol | 0.9 |
| Perfume | 0.2 |
| Glycerol | 8.7 |
| Methylparaben | 0.15 |
| Phenoxyethanol | 0.3 |
| Sodium Polyacrylate | 1.5 |
| Aqua | 74.2496 |

E (Example 2)

| INCI | % by weight |
|---|---|
| Olus Oil | 3.2 |
| Candelilla Cera | 0.2 |
| Cetyl Palmitate | 1 |
| Methyl Palmitate | 5 |
| Butyrospermum Parkii Butter | 4 |
| Hydrogenated Vegetable Oil | 0.6 |
| Zea Mays Oil + CI 40800 + Tocopherol | 0.0004 |
| Cyclomethicone + Dimethiconol | 0.9 |
| Perfume | 0.2 |
| Glycerol | 8.7 |
| Methylparaben | 0.15 |
| Phenoxyethanol | 0.3 |
| Sodium Polyacrylate | 1.5 |
| Aqua | 74.2496 |

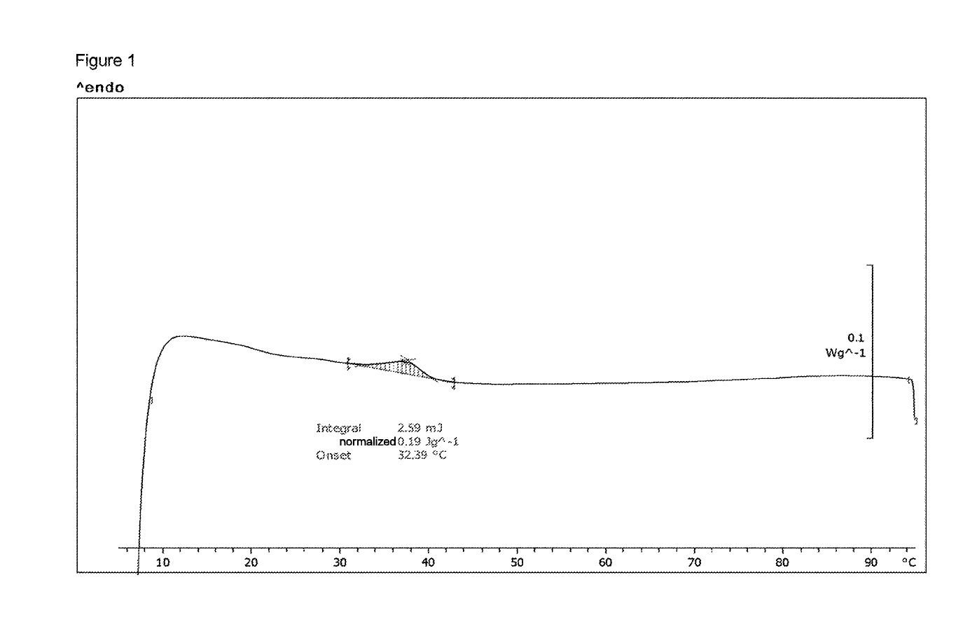

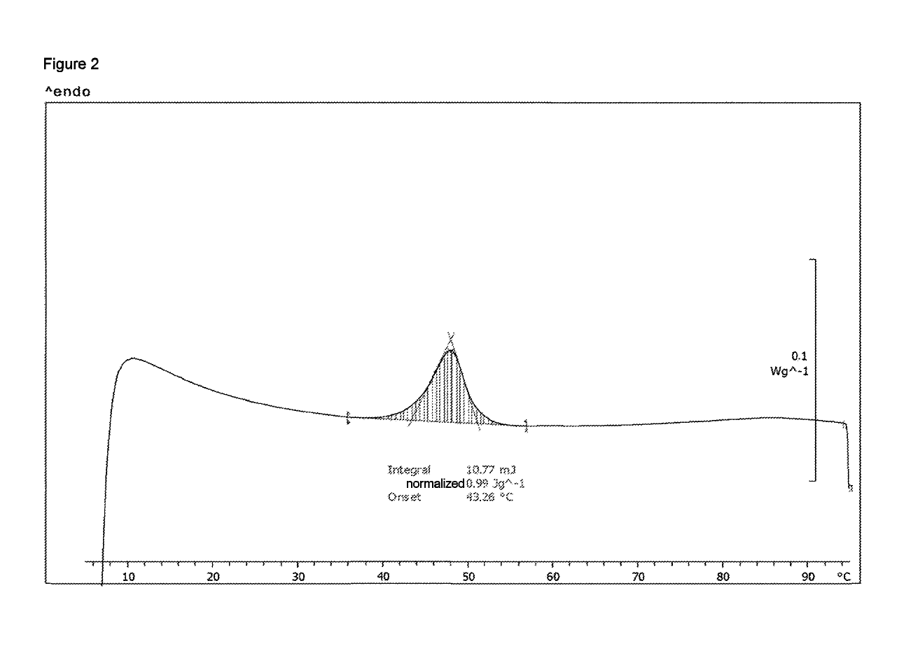

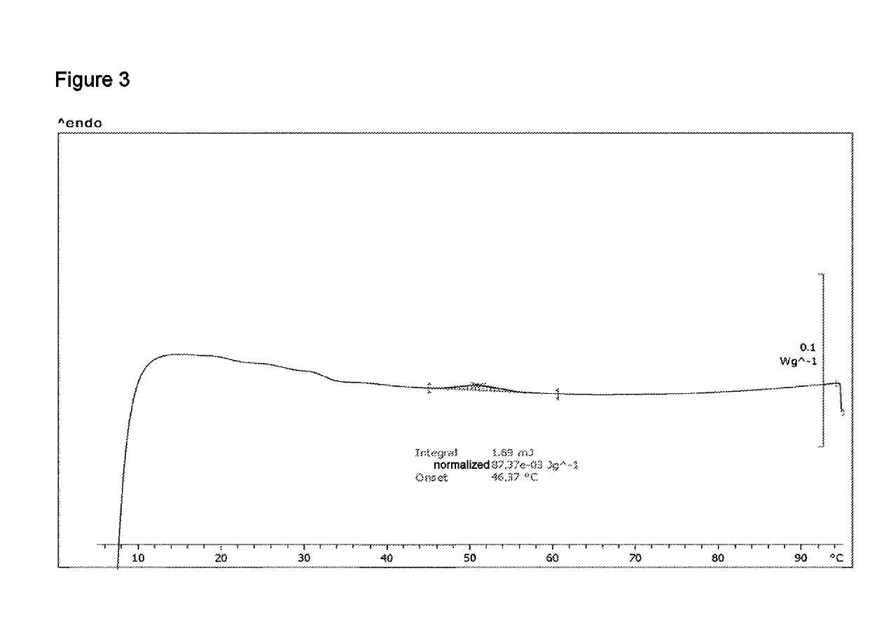

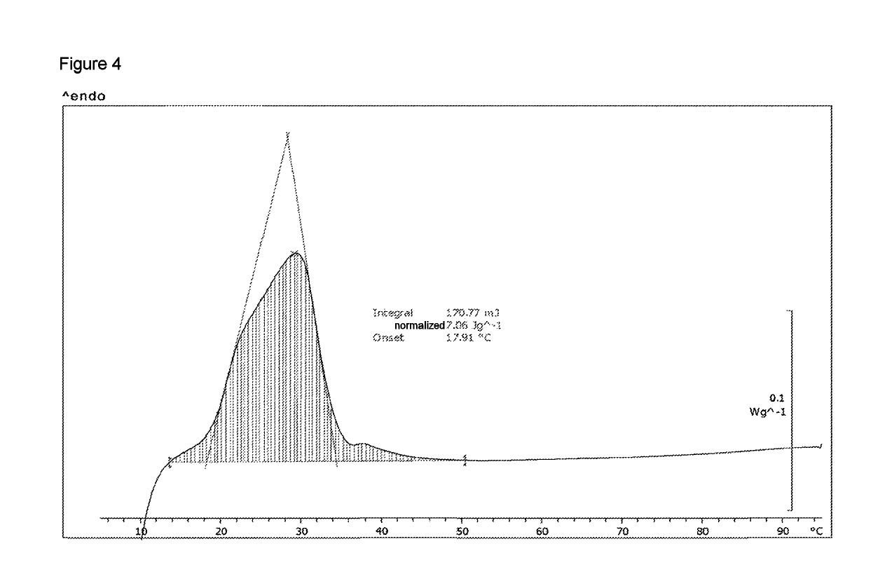

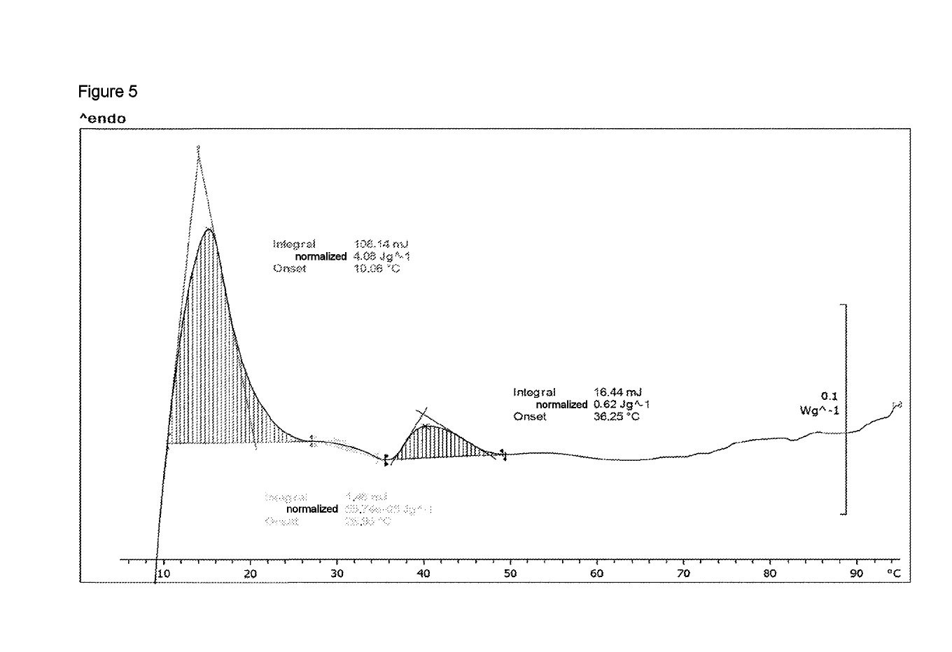

FIGS. 1 to 5 show the DSC analysis results of the formulations A to E.

The “endo” representation of the DSC measurements shown in FIGS. 1 to 5 enables positive (upward) depiction of the endothermic processes. The upward curves show that something is melting. The respective melt peaks are evaluated such that the enthalpy of fusion is shown (integral unnormalized and normalized), as is Tₒₙₛₑₜ as the melting temperature onset of the peak.

The prior art formulations A, B and C do not have any melting peaks between 0° C. and 30° C., meaning that the dispersions do not have a melting range between 5° C. and 30° C. by DSC.

In subsequent organoleptic tests, these formulations A, B, C were not found to be as pleasant as the inventive recipes D and E.

Inventive recipe D has a peak at 17.91° C. When applied to the skin, it feels very pleasant.

Inventive recipe E has several peaks, including 2 peaks between 0 and 30° C. When applied to the skin, it feels very pleasant.

The organoleptic assessment was made in the laboratory with n=4 trained subjects.

The inventive hydrodispersions are preferably cosmetic and/or dermatological formulations.

Stable cosmetic formulations are those which do not exhibit any kind of phase separation, change in viscosity, coalescence, Ostwald ripening and/or creaming over a prolonged period and in the event of a temperature change.

A phase separation of the formulation between the lipid phase and water phase, also referred to as breaking, is therefore undesirable during storage and use.

The formulations of the invention are stable and do not exhibit any phase separation at all on application to the skin.

The formulations of the invention are preferably free of anionic and/or cationic emulsifiers/surfactants.

If, however, anionic or cationic emulsifying substances or detersive substances are present in the formulations because of raw material impurities or other entrained materials, these formulations, in accordance with the invention, are still regarded as being “free of” emulsifiers and surfactants if the proportion thereof does not exceed 0.1% by weight, based on the total mass of the formulation.

Thickeners, which may under some circumstances also have emulsifying properties, however, are not counted among the emulsifiers that are to be excluded, since the main function thereof is not that of an emulsifier.

If restrictions to preferred proportional ranges of one or more ingredients are made and/or restrictions to particular ingredients, the preferred proportional ranges then relate to the selected ingredients.

The inventive hydrodispersion may, as well as the waxes, also contain other oils or lipids, advantageously in a proportion of up to 10% by weight, based on the total mass of the formulation. However, it should be ensured that the overall formulation should have at least one melting range within the inventive range from 5° C. to 30° C.

This can be achieved by choosing inventive waxes having a Tₒₙₛₑₜ<30° C., and by correspondingly testing the dispersions obtained.

It is advantageous that the total amount of the waxes is greater than the total amount of the oils.

Additional lipids or oils are preferably chosen from the group of the polar oils, for example from the group of the lecithins and the fatty acid triglycerides, namely the triglyceryl esters of saturated and/or unsaturated, branched and/or unbranched alkanecarboxylic acids having a chain length of 8 to 24 and especially 12 to 18 C atoms. The fatty acid triglycerides may advantageously be chosen, for example, from the group of the synthetic, semisynthetic and natural oils, for example cocoglyceride, olive oil, sunflower oil, soya oil, peanut oil, rapeseed oil, argan oil, almond oil, palm oil, coconut oil, castor oil, wheatgerm oil, grapeseed oil, safflower oil, evening primrose oil, macadamia nut oil and the like.

Further advantageous polar oil components in the context of the present invention may also be selected from the group of the esters of saturated and/or unsaturated, branched and/or unbranched alkane-carboxylic acids having a chain length of 3 to 30 C atoms and saturated and/or unsaturated, branched and/or unbranched alcohols having a chain length of 3 to 30 C atoms, and from the group of the esters of aromatic carboxylic acids and saturated and/or unsaturated, branched and/or unbranched alcohols having a chain length of 3 to 30 C atoms. Such ester oils can then advantageously be selected from the group of octyl palmitate, octyl cocoate, octyl isostearate, octyldodecyl myristate, octyldodecanol, cetearyl isononanoate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, stearyl heptanoate, oleyl oleate, oleyl erucate, erucyl oleate, erucyl erucate, tridecyl stearate, tridecyl trimellitate, and also synthetic, semisynthetic and natural mixtures of such esters, for example jojoba oil.

In addition, the oil phase can advantageously be chosen from the group of the dialkyl ethers and dialkyl carbonates, advantageous examples being dicaprylyl ether (Cetiol OE) and/or dicaprylyl carbonate, for example that obtainable under the Cetiol CC trade name from Cognis.

In addition, the oil phase may likewise advantageously also contain nonpolar oils, for example those which are selected from the group of the branched and unbranched hydrocarbons, especially mineral oil, Vaseline (petrolatum), paraffin oil, squalane and squalene, polyolefins, hydrogenated polyisobutenes and isohexadecane. Among the polyolefins, polydecenes are the preferred substances.

Preference is given to natural oil, vegetable oil or mineral oil, especially paraffinum liquidum, especially in a proportion of 2% to 4% by weight in each case, based on the total mass of the formulation.

The formulations of the invention may optionally have content of cyclic and/or linear silicone oils.

Advantageously, cyclomethicones are used, especially octamethylcyclotetrasiloxane, cyclomethicone D5 and/or cyclomethicone D6, as silicone oil of the invention. A further silicone oil which is advantageous in accordance with the invention is dimethicone (also: dimethylpolysiloxane or polydimethylsiloxane with the INCI name Dimethicone).

Other silicone oils can also be used advantageously in the context of present invention, for example hexamethylcyclotrisiloxane, poly(methylphenylsiloxane), phenyltrimethicone, phenyldimethicone, hexamethylcyclotrisiloxane, polydimethylsiloxane, poly(methyl-phenylsiloxane), cetyldimethicone, behenoxydimethicone.

It is also advantageous to choose silicone oils of similar constitution to the compounds described above, wherein the organic side chains have been derivatized, for example polyethoxylated and/or polypropoxylated. Examples of these include polysiloxane polyalkyl polyether copolymers such as cetyl dimethicone copolyol or else lauryl methicone copolyol.

A particularly advantageous mixture used for achievement of the texture of the invention is a mixture of cyclic and linear silicone oils, especially of cyclomethicone and dimethicone.

The proportion of one or more silicone oils, more preferably cyclomethicone and dimethicone, is preferably chosen within the range from 0.1% to 10% by weight, particularly 0.5% to 8% by weight, based in each case on the total weight of the formulation.

In a particularly preferred embodiment, the formulations in the context of the present invention contain what are called moisturizers. Moisturizers refer to substances or substance mixtures which endow cosmetic formulations with the property, after application or distribution on the skin surface, of reducing the release of moisture from the horny layer (also called transepidermal water loss (TEWL)) and/or of positively influencing the hydration of the horny layer.

Advantageous moisturizers in the context of present invention are, for example, glycerol, lactic acid and/or lactates, especially sodium lactate, butylene glycol, propylene glycol, Biosaccharide Gum-1, glycine soja, ethylhexyloxyglycerol, pyrrolidonecarboxylic acid and urea, Glyceryl Glucoside.

Advantageously, the formulation of the invention comprises Glyceryl Glucoside, especially in a proportion of 0.001% to 8% by weight, based on the total mass of the formulation.

Advantageously, the amount of moisturizers, one or more compounds, is chosen from the range from 1% to 20% by weight, preferably from 3% to 15% by weight, more preferably from 5% to 12% by weight—based in each case on the total weight of the formulation.

The cosmetic formulations in the context of the present invention may, according to their makeup, be used, for example, as skin protection cream, cleansing milk, sunscreen solution, nutrient cream, day or night cream, etc. Particular preference is given in the context of the present invention to formulations for application, especially over a large area, in the personal care sector.

Advantageously, the hydrodispersion or formulation is used as skincare cream.

The advantages of the hydrodispersion of the invention are the ease of distribution thereof, the refreshing effect on application and nevertheless a care effect which is caused especially by the waxes.

The hydrodispersion of the invention is particularly skin-compatible, since a low level of oils, if any, is present, and especially no spreading oils, a low level of emulsifiers and a low level of preservatives. Application of the hydrodispersion to the eye is therefore possible without any problem.

The compositions may also contain UV filters selected from the group of the compounds 2-phenylbenzimidazole-5-sulfonic acid and/or salts thereof, phenylene-1,4-bis-(2-benzimidazoyl)-3,3′,5,5′-tetrasulfonic salts; 1,4-di(2-oxo-10-sulfo-3-bornylidenemethyl)benzene and salts thereof; 4-(2-oxo-3-bornylidenemethyl)benzenesulfonic salts; 2-methyl-5-(2-oxo-3-bornylidenemethyl)sulfonic salts; 2,2′-methylenebis-(6-(2H-benzotriazol-2-yl)-4-(1,1,3,3-tetramethylbutyl)phenol); 2-(2H-benzotriazol-2-yl)-4-methyl-6-[2-methyl-3-[1,3,3,3-tetramethyl-1-[(trimethylsilyl)oxy]disiloxanyl]propyl]phenol; 3-(4-methylbenzylidene)camphor; 3-benzylidenecamphor; ethylhexyl salicylate; terephthalidenedicamphorsulfonic acid; 4-(tert-butyl)-4′-methoxydibenzoylmethane; 2-ethylhexyl 2-cyano-3,3-diphenylacrylate; 2-ethylhexyl 4-(dimethylamino)benzoate; Diethylamino Hydroxybenzoyl Hexyl Benzoate, amyl 4-(dimethylamino)benzoate; di(2-ethylhexyl 4-methoxybenzalmalonate; 2-ethylhexyl 4-methoxycinnamate; isoamyl 4-methoxycinnamate; 2-hydroxy-4-methoxybenzophenone, 2-hydroxy-4-methoxy-4′-methylbenzophenone; 2,2′-dihydroxy-4-methoxybenzo-phenone; hexyl 2-(4′-diethylamino-2′-hydroxybenzoyl)benzoate; homomenthyl salicylate; 2-ethylhexyl 2-hydroxybenzoate; dimethicodiethyl benzalmalonate; 3-(4-(2,2-bisethoxycarbonylvinyl)phenoxy)propenyl)methoxysiloxane/dimethylsiloxane copolymer; dioctylbutylamidotriazone (INCI: Diethylhexyl Butamidotriazone); 2,4-bis[5-(1-dimethylpropyl)-benzoxazol-2-yl-(4-phenyl)imino]-6-(2-ethylhexyl)-imino-1,3,5-triazine having CAS No. 288254-16-0; tris(2-ethylhexyl) 4,4′,4″-(1,3,5-triazine-2,4,6-triyltriimino)trisbenzoate) (also: 2,4,6-tris[anilino-(p-carbo-2′-ethyl-1′-hexyloxy)]-1,3,5-triazine (INCI: Ethylhexyl Triazone); 2,4-bis {[4-(2-ethylhexyloxy)-2-hydroxy]phenyl}-6-(4-methoxyphenyl)-1,3,5-triazine; 2,4,6-tribiphenyl-4-yl-1,3,5-triazine; merocyanines, butyl methoxydibenzoylmethane, octocrylene, and titanium dioxides and zinc oxides.

The cosmetic or dermatological formulations according to the invention may also contain cosmetic auxiliaries and active ingredients as typically used in such formulations, for example preservatives, preservation aids, bactericides, substances for preventing foaming, dyes and color pigments, thickeners, moistening and/or moisturizing substances, anti-ageing substances or other customary constituents of a cosmetic or dermatological formulation such as polyols, polymers, foam stabilizers, organic solvents or silicone derivatives, provided that the addition does not impair the required properties in terms of organoleptics and care effects. Active ingredients present may, for example, be chosen from the group of the following compounds: glycyrrhetinic acid, urea, arctiin, alpha-lipoic acid, folic acid, phytoene, D-biotin, coenzyme Q10, alpha-glucosylrutin, tocopheryl acetate, carnitine, carnosine, caffeine, natural and/or synthetic isoflavonoids, glycerylglucose, creatine, creatinine, taurine, magnolia, β-alanine and/or licochalcone A.

The examples which follow elucidate the hydrodispersions of the invention. The numbers cited are, unless stated otherwise, proportions by weight based on the total mass of the formulation.

### EXAMPLES 1 AND 2

| INCI | Recipe 1 | Recipe 2 |
|---|---|---|
| Olus Oil | 3.2 | 3.2 |
| Candelilla Cera | 0.2 | 0.2 |
| Cetyl Palmitate | 1 | 1 |
| Methyl Palmitate | 5 | 0 |
| Hydrogenated Coco-Glycerides | 0 | 5 |
| Butyrospermum Parkii Butter | 4 | 4 |
| Hydrogenated Vegetable Oil | 0.6 | 0.6 |
| Cyclomethicone + Dimethiconol | 0.9 | 0.9 |
| Perfume | 0.2 | 0.2 |
| Glycerol | 8.7 | 8.7 |
| Zea Mays Oil + CI 40800 + Tocopherol | 0.0004 | 0.0004 |
| Methylparaben | 0.15 | 0.15 |
| Phenoxyethanol | 0.3 | 0.3 |
| Sodium Polyacrylate | 1.5 | 1.5 |
| Aqua | 74.2496 | 74.2496 |

The hydrodispersions of the invention exhibit a pleasant, sensorily light profile and good care effects. In addition, the formulations do not break on application to the skin.

1.-15. (canceled)

16. A hydrodispersion, wherein the hydrodispersion comprises, based on a total mass of the dispersion:
(a) water,
(b) from 0.05% to 5% by weight of at least one thickener,
(c) from 2% to 20% by weight of one or more waxes, at least one wax having a melting point Tₒₙₛₑₜ below 30° C., and
(d) from 0% to 20% by weight of one or more oils.

17. The hydrodispersion of claim 16, wherein the hydrodispersion exhibits at least one melting range between 5° C. and 30° C. by DSC.

18. The hydrodispersion of claim 16, wherein (b) comprises at least one substance selected from homopolymers or copolymers of acrylic acid and/or acrylamide and derivatives thereof, organic natural compounds, organic modified natural substances, organic fully synthetic compounds.

19. The hydrodispersion of claim 18, wherein (b) comprises at least one substance selected from homopolymers or copolymers of acrylic acid and/or acrylamide and derivatives thereof, agar-agar, carrageenan, tragacanth, gum arabic, alginates, pectins, polyoses, guar flour, carob seed flour, starch, dextrins, gelatins, casein, xanthan gum, carboxymethyl cellulose, hydroxyethyl and/or hydroxypropyl cellulose, microcrystalline cellulose, polyacrylic and/or polymethacrylic compounds, vinyl polymers, polycarboxylic acids, polyethers, polyimines, polyamides, polyurethanes, AMPS copolymers, acrylic acid/VP Crosspolymer, PVP, acrylamide/ammonium acrylate copolymer.

20. The hydrodispersion of claim 16, wherein (b) comprises sodium polyacrylate.

21. The hydrodispersion of claim 16, wherein from 0.1% to 2% by weight of (b) are present.

22. The hydrodispersion of claim 16, wherein from 60% to 95% by weight of (a) are present.

23. The hydrodispersion of claim 21, wherein from 70% to 85% by weight of (a) are present.

24. The hydrodispersion of claim 16, wherein the at least one wax having a Tₒₙₛₑₜ below 30° C. is selected from fats (triglycerides), mono- and diglycerides, natural and synthetic waxes, fatty alcohols, wax alcohols, fatty acids, esters of fatty alcohols and fatty acids, fatty acid amides, and any combinations of these substances.

25. The hydrodispersion of claim 16, wherein the at least one wax having a Tₒₙₛₑₜ below 30° C. comprises at least one of Shorea stenoptera seed butter, hydrogenated vegetable oil, hydrogenated coco-glycerides, Butyrospermum parkii butter, Theobroma cacao (cocoa) seed butter, mango butter, methyl palmitate, hydrogenated palm kernel glycerides, hydrogenated palm glycerides, Acacia decurrens/jojoba/sunflower seed wax polyglyceryl-3 esters, cetearyl nonanoate, cetearyl alcohol.

26. The hydrodispersion of claim 16, wherein the hydrodispersion comprises more than 0.5% by weight of (d).

27. The hydrodispersion of claim 26, wherein the hydrodispersion comprises from 1% to 10% by weight of (d).

28. The hydrodispersion of claim 26, wherein a concentration of (c) is greater than a concentration of (d).

29. The hydrodispersion of claim 16, wherein the hydrodispersion does not contain any anionic emulsifiers or surfactants.

30. The hydrodispersion of claim 16, wherein the hydrodispersion does not contain any cationic emulsifiers or surfactants.

31. The hydrodispersion of claim 16, wherein the hydrodispersion does not contain any anionic or cationic emulsifiers or surfactants.

32. A cosmetic or dermatological composition, wherein the composition comprises or consists of the hydrodispersion of claim 16.

33. The composition of claim 32, wherein the composition is a skincare cream.

34. The composition of claim 32, wherein the composition is an eye cream.

35. A method of skincare, wherein the method comprises applying to skin in need of skincare the composition of claim 32.
